(19) United States
(12) Patent Application Publication
(10) Pub. No.: US 2015/0150724 A1
(43) Pub. Date: Jun. 4, 2015

(54) GOGGLES WITH INTERCHANGEABLE LENS

(71) Applicant: ELECTRIC VISUAL EVOLUTION, LLP, San Clemente, CA (US)

(72) Inventor: Kip Arnette, San Clemente, CA (US)

(21) Appl. No.: 14/558,040

(22) Filed: Dec. 2, 2014

Related U.S. Application Data
(60) Provisional application No. 61/911,357, filed on Dec. 3, 2013.

(51) Int. Cl. A61F 9/02

(52) U.S. Cl. CPC A61F 9/02

(57) ABSTRACT

The invention generally relates to a goggle with an interchangeable lens. In certain aspects, a goggle system of the invention includes a frame defining and opening and comprising a forward surface that circumscribes the opening. The forward surface includes a groove element along the entirety of the forward surface. The removable lens includes a gasket configured to mate with the grove element of the frame to form a continuous seal between the frame and the removable lens.

### RELATED APPLICATION

This application claims the benefit of and priority to U.S. Provisional No. 61/911,357, filed Dec. 3, 2013, which is incorporated by reference in its entirety.

### TECHNICAL FIELD

The invention generally relates to a goggle system with an interchangeable lens.

### BACKGROUND

Outdoor activities, such as skiing, snowboarding, and alpine hiking, often require a user to wear goggles. Goggles for those activities are also referred to as ski or snow goggles. The type of lens that a user requires for a certain activity can vary depending on the weather conditions and time of day. As such, goggle lenses can come in a variety of different tints in order to optimize visibility and comfort. For example, different tints provide varying degrees of the amount of visual light that transmits through the lens. The amount of visual light transmitted through a lens is referred to as visual light transmittance (VLT). A lower degree of VLT allows less light to pass through the lens making a low VLT lens more suitable for sunny conditions, and a higher degree of VLT allows increased amounts of light through the lens making a high VLT lens more suitable for low light (darker) conditions.

In order to accommodate for the various light conditions, some manufacturers have created goggles with interchangeable lens. These goggles allow a user to replace a lens of a goggle depending on the activity. Currently, there are two main types of interchangeable lens goggles. The first type requires removing a lens from a slot within the frame, and then sliding a new lens within the slot. However, successfully inserting the lens into the slot is a difficult and time consuming task. Another type of interchangeable lens goggles includes one or more levers that must be released to remove a lens, and the lever must be re-engaged to hold a new lens in. Often the lever undesirably encroaches or limits the field of vision of the lens. In addition, manipulating the levers to remove and re-secure the lens can prove difficult, and often requires a user to remove the goggle from their face in order to do so.

Because of the above difficulties with current goggles with an interchangeable lens, users are not able to successfully interchange the lens while in the middle of an activity or during adverse conditions. Adverse conditions may include cold, rain, ice, snow, darkness, as well as when a user is wearing gloves (such as ski or hiking gloves). As a result, current goggles with interchangeable lenses are not practical for use in the field.

### SUMMARY

The present invention generally relates to a goggle system that allows a user to quickly and easily change a lens as needed during use and/or under less than ideal conditions. The goggle system includes a frame and a removable lens. The goggle system may also include a gasket that allows the lens to couple with a groove on the frame. The gasket may be coupled to the lens or formed as part of the lens. A benefit of the goggle system is that the lens may be sealed to the frame by simply pressing the lens against the frame such that the gasket of the lens mate-fits with the groove on the frame. Likewise, a lens may be removed from the goggle system by simply applying pressure on the lens away from the goggle frame. With the press-fit mechanism, goggles of the invention allow a user to easily change the lens regardless of the activity and conditions. Thus, goggles of the invention allow user to quickly change lenses for optimal visibility without the hassle of prior art goggles.

According to certain aspects, a goggle system of the invention includes a frame and a removable lens. The frame defines an opening and includes a forward surface that circumscribes the opening. The forward surface includes a groove element along the entirety of the forward surface. The removable lens includes a gasket that is configured to mate with the groove element of the frame to form a continuous seal between the frame and the removable lens. For example, the removable lens may couple to the frame via a tongue and groove junction. The continuous seal provides a 360° point of contact seal between the frame and the lens. The continuous seal securely fastens the lens when coupled to the frame, and eliminates all penetrating debris, wind, water, and other particulates from entering the goggles during use.

In further aspects, a goggle system of the invention includes a frame and a lens. The frame defines an opening and includes a forward surface that circumscribes the opening. The forward surface faces away from a user's face when wearing the goggles system and comprises an outer edge around the entirety of the forward surface. The lens is configured to removably couple to the frame such that an outer edge of the lens is substantially flush with the outer edge of the frame. The lens may removably couple to the frame using a tongue and grove junction.

In certain embodiments, the lens couples to the frame using a tongue and groove junction between the gasket of the lens and the frame. The lens may include a tongue element, and the frame may include a groove element. The tongue element may include a protrusion that enters a channel of the groove element. A first portion of the protrusion may include a ledge member. The ledge member acts to secure the gasket (and thus lens) to the frame. In addition, a second portion of the protrusion does not include a ledge member. The second portion without a ledge member forms a removal point between the lens and the frame when coupled. The removal point is a point in the seal between the frame and the gasket that require less pressure to remove the lens than a point in the seal between the frame and the gasket where the protrusion has a ledge member.

### BRIEF DESCRIPTION OF DRAWINGS

FIG. 1 illustrates an angled view of a goggle system of the invention, according to certain aspects.

FIG. 2 illustrates a top view of a goggle system of the invention, according to certain aspects.

FIG. 3 illustrates a bottom view of a goggle system of the invention, according to certain aspects.

FIG. 4 illustrates an exploded view of a goggle system of the invention, according to certain aspects.

FIG. 5 illustrates another bottom view of a goggle system of the invention, according to certain aspects.

FIG. 6A illustrates a side view of a gasket of the goggle system, according to certain aspects.

FIG. 6B illustrates a back view of a gasket of the goggle system, according to certain aspects.

FIG. 6C illustrates a cross-sectional view of a gasket of the goggle system, according to certain aspects.

FIG. 6D illustrates another cross-sectional view of a gasket of the goggle system, according to certain aspects.

FIG. 7 illustrates a cross-sectional view of the goggle system with a lens mated to a frame, according to certain aspects.

### DETAILED DESCRIPTION

The present invention generally relates to a goggle system with an interchangeable lens. The goggle system includes a goggle frame and a removable lens. The lens of the goggle system may further include a gasket that allows for the lens to couple with the goggle frame. The gasket may be coupled to the lens or formed as part of the lens. A benefit of the goggle system is that the lens may be sealed to the frame by simply pressing the lens against the goggle frame such that the gasket of the lens mate fits with a groove on the frame. As described in more detail, the mate fit of the gasket of the lens with the groove on the goggle frame forms a continuous seal between the goggle frame and the lens. As a result, the goggle system of the invention eliminates or minimizes penetrating debris, wind, water, and other particulates from entering the goggles during use. The goggle system of the invention is ideal for use during a variety of activities including, for example, skiing, snowboarding, alpine climbing, motorcycling, using an all-terrain vehicle, and during watersport activities—such as waterskiing and jetskiing.

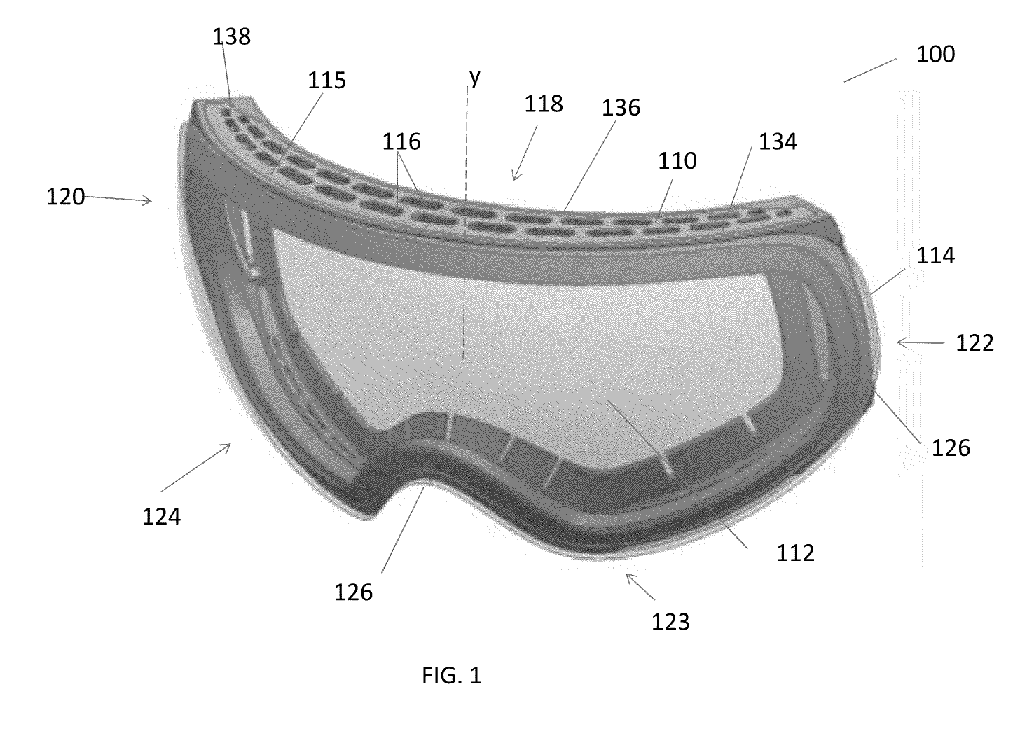

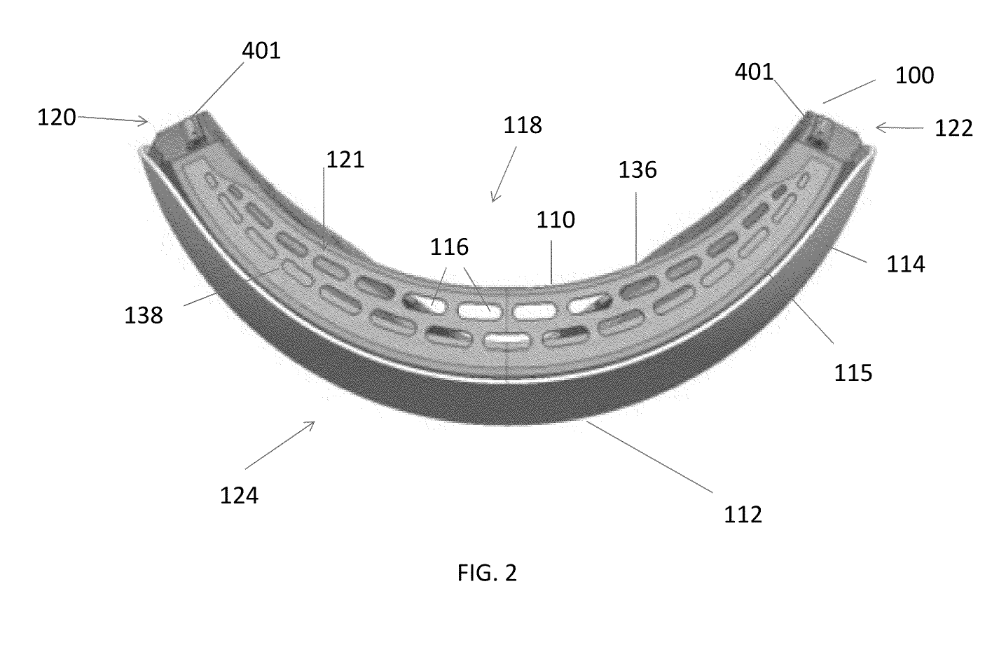

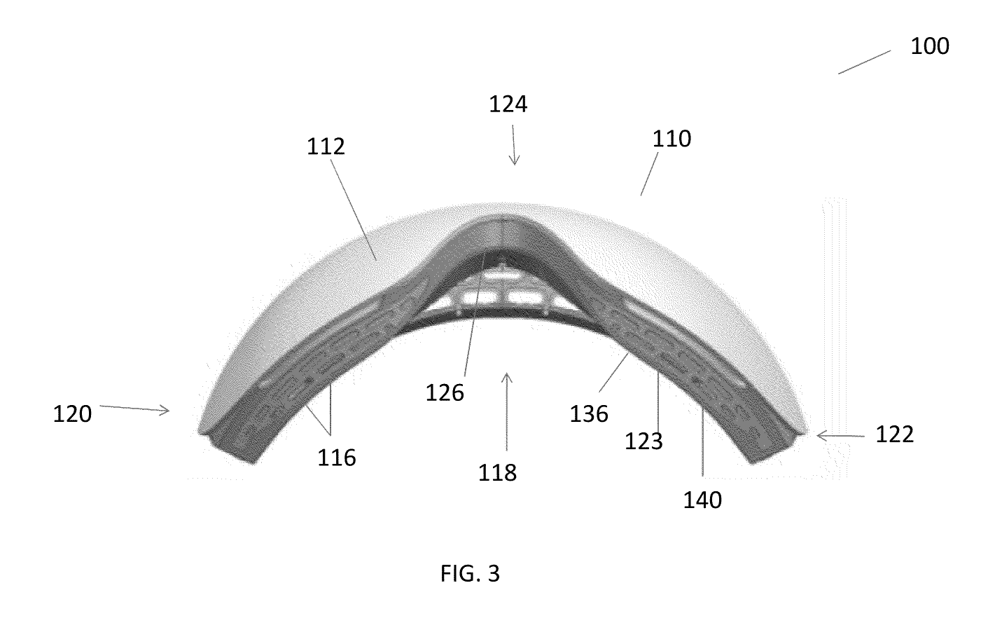

FIG. 1 illustrates an angled front view of the goggle system 100 of the invention. The goggle system includes a front 124, a back 118, top 121, bottom, 123, a first side 120, and a second side 122. FIG. 2 depicts a top view of the goggle system 100, and FIG. 3 depicts a bottom view of the goggle system.

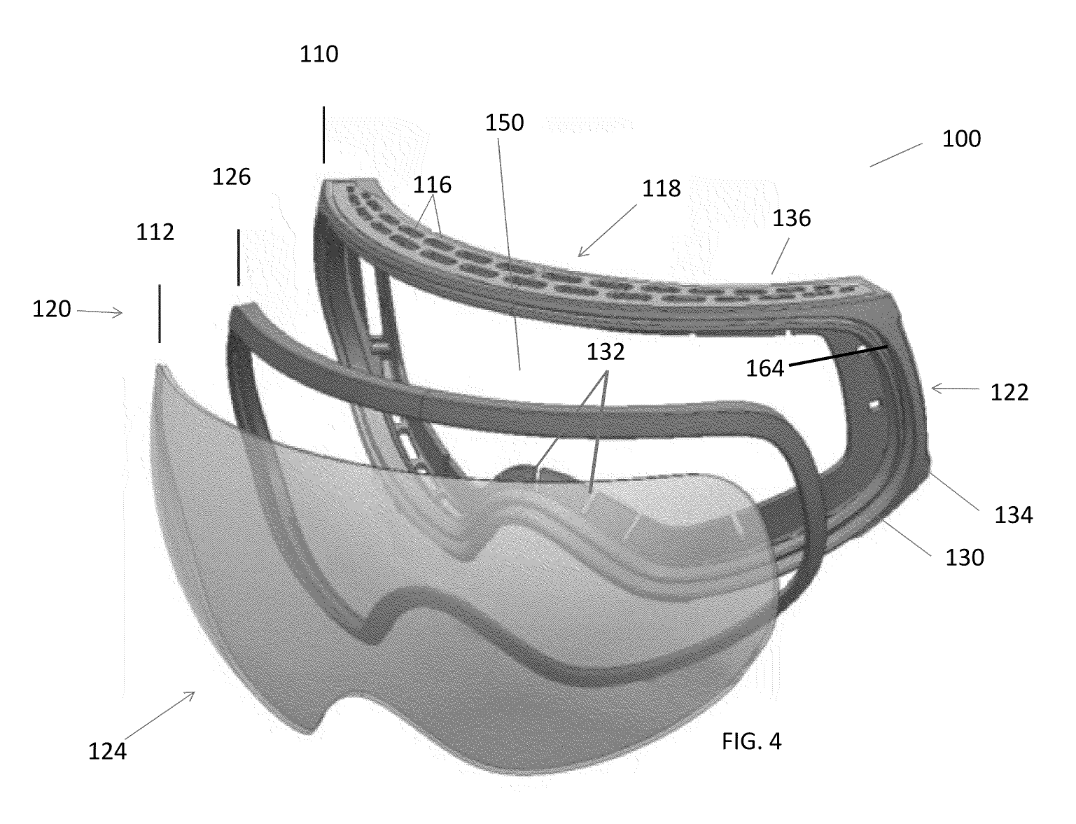

As shown in FIG. 1, the goggle system 100 includes a lens 112 and a goggle frame 110. The lens 112 is removably coupled to the frame 110 using a mate-fit coupling (described hereinafter). The frame 110 is designed to fit on a face of a user and defines an opening 150 (See FIG. 4). In certain embodiments, the frame 110 and the lens 112 are curved to conform with the curvature of a user's face. The curvature of the frame 110 and lens 112 is best illustrated in FIGS. 2 and 3, which depict top and bottom sides of the goggle system 100. The radius of curvature can vary depending on whether the goggle system is made for small, medium, and large faces, certain genders (male or female), adults, or for children. Alternatively, the frame 110 may be designed such that a back surface 136 of the frame 110 conforms with the curvature of a user's face, while the front surface 134 of the frame and the lens 112 are flat. The frame 110 and the lens 112, as shown, include a recess 126 that allows the goggle system 100 to rest above a nose of the user. Alternatively, the goggle system 100 can be designed to fit over the nose of a user.

When the lens 112 is coupled to the frame, the opening 150 (see FIG. 4) of the frame 110 is covered by the lens 112. The opening 150 provides a user with an unobstructed view through lens 112 when the lens 112 is coupled to the frame 110.

The frame 110 includes a front surface 134 and a back surface 136. When the lens 112 and frame 110 are coupled together, a portion of the back surface of the lens 112 is placed on top of the front surface 134 of the frame 110. The back surface 136 of the frame rests against a user's face when wearing the goggle system. The front surface 134 of the frame faces away from a user's face when wearing the goggle system. The front surface 134 of the frame includes an outer edge 115 (i.e. perimeter), and the lens includes an outer edge 114 (i.e. perimeter). According to certain aspects, the front surface 134 of the frame 110 is configured to mate with the removable lens 112 such that the outer edge 114 of the lens 112 is substantially flush with the outer edge 115 of the frame 110. In other words, the outer perimeter of the lens 112 is substantially flush with the outer perimeter of the frame. This configuration optimally maximizes the unobstructed size/amount of the lens 112 that a user can use to see through because the lens 112 is not coupled to an inner surface of the frame 110. In addition, this configuration provides a streamline look because the body of the frame 110 is substantially not visible when looking at the front 124 of the goggle system 100.

According to certain embodiments, the frame 110 further includes one or more vent openings 116. Preferably, the vent openings 116 are located on a top surface 138 and/or bottom surface 140 of the frame 110 (See FIGS. 2 and 3). The vent openings 116 allow air to enter and leave the frame 110 for breathability. In one embodiment, a filter element, such as a porous mesh or foam, is placed over the vent openings 116 in order to ensure that particulates do not enter through the vent openings 116.

The goggle system 100 can be designed to operate with any strap(s), bands, or other mechanism to hold the goggle system 100 onto the head of a user. The strap may be elastic, adjustable, or both. FIG. 2 illustrates connectors 401 within the frame 110 that can couple to and hold one or more straps.

FIG. 4 illustrates an exploded view of the goggle system 100 according to certain aspects. As shown in FIG. 4, the goggle system 100 includes a frame 110, a lens 112 and a gasket 126. As shown in the exploded view, the gasket 126 is a separate piece that can be coupled to the lens 112. However, the gasket 126 can be formed as part of the lens 112. The gasket 126 (when either separate from or formed as part of the lens 112) is also referred to herein as a protrusion to the lens 112.

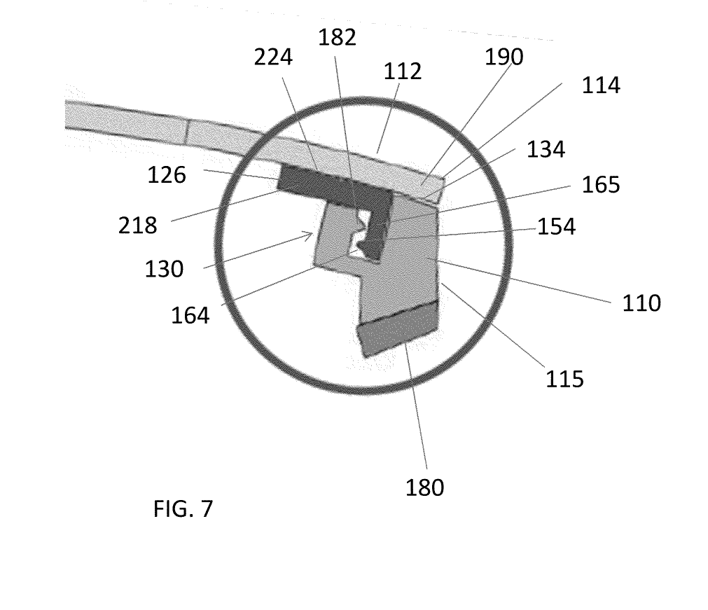

As shown in FIG. 4, the frame defines an opening 150 and includes a front surface 134 that circumscribes/surrounds the opening 150. The front surface 134 of the frame 110 includes a groove element 130. The groove element 130 extends along the entirety of the front surface 134, and thus also circumscribes the opening 150. The groove element 130 forms a channel 164 in the frame and is configured to receive and couple with the gasket 126 of the lens. In certain embodiments, the groove element includes a tab 182 (described in more detail in reference to FIG. 7). The tab 182 secures the gasket within the channel 164 of the groove element 130.

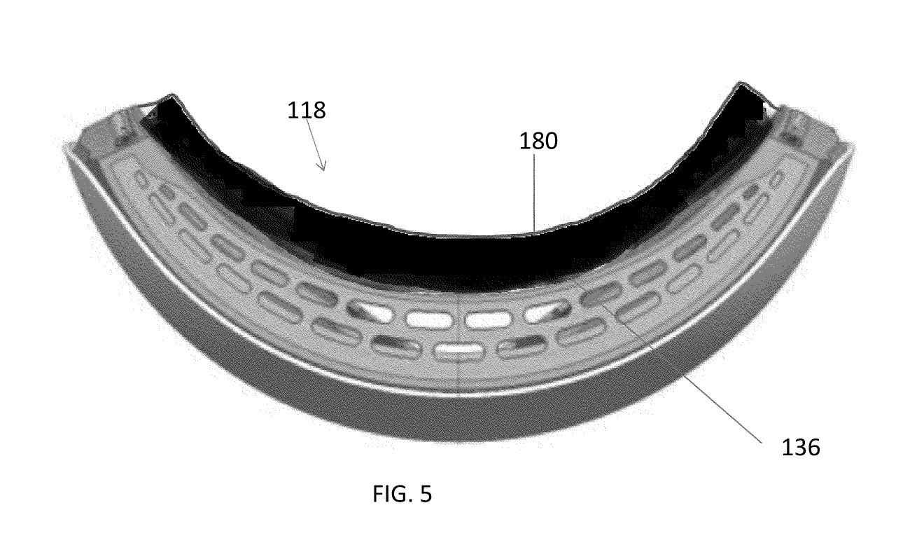

The frame 110 also includes a back surface 136 that circumscribes the opening. The back surface 136 interfaces with a user's face while wearing the goggle system 110. The back surface 136 may optionally include cutouts 132. The cutouts 132 provide flexibility to the goggle system 100 because they allow the frame 110 to remain flush against a user face regardless of the user's facial expression or regardless of the pressure being exerted on the frame (the pressure being exerted, for example, by wind or by one or more straps holding the goggle system to the user's face). In certain embodiments, the back surface 136 of the frame 110 is coupled to a cushion 180. The cushion 180 is shown in FIG. 5. The cushion 180 rests against the face of a user while wearing the goggle system, and provides comfort. The cushion 180 can be made of any material. The cushion 180 may surround the entirety of the back surface 136 or a portion of the back surface 136. Like the back surface 136, the cushion 180 may circumscribe the opening 150 of the frame 110. Preferably, the cushion 180 is formed from a breathable foam padding (e.g., a porous padding). The cushion 180 can be made of one or more different layers of padding. The padding can vary in density and strength. For example, a denser padding layer may be coupled directly to the frame 110, while a less dense padding layer is formed on top of the denser padding layer. The face of the user wearing the goggle system may then rest against the less dense padding layer. Preferably, the cushion 180 is placed over the flexible cutouts 132 on the back surface 136. This prevents the cutouts 132 from rubbing against or annoying the user, while maintaining the flexibility and function of the cutouts 132.

As discussed, the gasket 126 may be coupled to the lens 112 or the gasket 126 may formed as part of the lens 112. The gasket 126 may be coupled to the lens 112 using any suitable manner. For example, the gasket 126 may be separate piece that is co-molded into the lens 112 during formation of the lens 112. In other embodiments, the gasket 126 is coupled to the lens 112 using an adhesive. Any adhesive may be used to couple the gasket 126 to the lens 112. The gasket 126 is used to form a releasable seal between the lens 112 and the frame 110. Preferably, the body of the lens 112 overhangs from the gasket 126 coupled thereto so that a portion of the lens 112 extends over a portion of the frame 110 when the gasket 126 is coupled to the frame 110. The overhang 190 of the lens 112 covering the frame 110 is best shown in FIG. 7, which depicts a cross-section y of the goggle system 100. The lens overhang 190 ensures that the outer edge/perimeter 114 of the lens 112 is substantially flush with the outer edge/perimeter 115 of the frame 110.

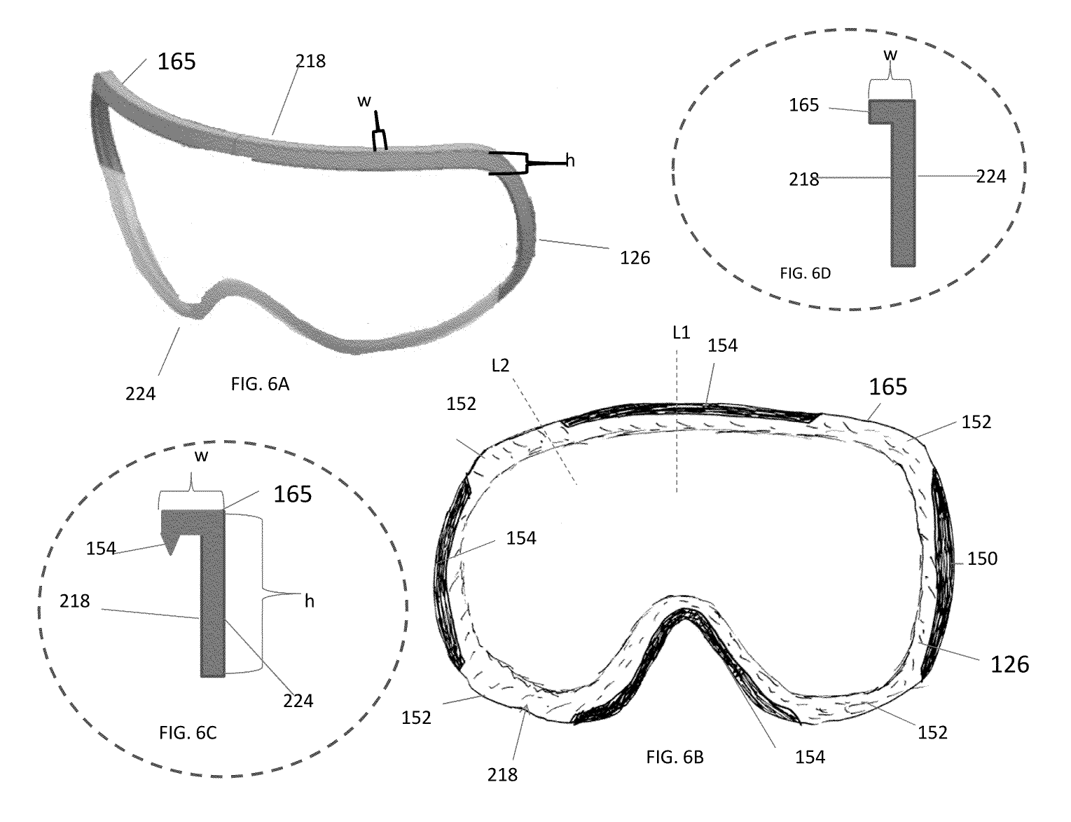

The gasket 126 is shown in more detail in FIGS. 6A-6D. FIG. 6A illustrates an angled view of the front surface 224 of the gasket 126. The gasket includes a width and a height (see FIGS. 6A, 6B). The front surface 224 of the gasket 126 couples to the lens 112, according to certain embodiments. FIG. 6B depicts a view of the back surface 218 of the gasket 126. In certain embodiments, a second lens is coupled to the back surface 218 of the gasket 126. In these embodiments, the goggle system is a duel-lens system. In duel-lens systems, a main lens (such as lens 112) may be coupled to a front surface of the gasket, and a second lens may fit within and couple to a back surface of the gasket. The second lens is typically smaller than the main lens. The back surface 218 of the gasket 126 is configured to mate with the groove element 130 of the frame 110. In preferred aspects, the mate-fit of the gasket 126 and the groove element 130 of the frame 110 forms a continuous seal. A continuous seal is a seal that has a 360° point of contact coupling between the frame 110 and the gasket 126.

Preferably, the gasket 126 and the frame 110 releasably couple together with a tongue and groove junction. In such embodiment, the gasket 126 includes a tongue element that mates with the groove element 130 of the frame's front surface 134. A benefit of a tongue and groove junction is that one may couple the lens 112 to the frame 110 by simply pressing the tongue of the gasket 126 into the groove element 130 of the frame 110. Because the goggle system 100 has a press-fit coupling, the goggle system 100 of the invention advantageously does not require a clasp to hold the interchangeable lens in place.

In certain embodiments, tongue element of the gasket 126 includes a protrusion 165. As shown in FIG. 6A-6D, the protrusion 165 extends distally from the outer edge of the gasket back surface 218. Thus, in this embodiment, the protrusion 165 forms the outer width (w) of the gasket 165. However, the protrusion 165 may extend from anywhere on the back surface 218 of the gasket 126. Preferably, the protrusion 165 is formed along the entirety of the back surface 218 of the gasket so that the protrusion 165 enters the channel 164 of the groove element 130 to form a continuous point of contact seal between the gasket 126 and the frame 110 (e.g. 360° seal). In other words, the protrusion 165 enters the entire of length of the channel 164 of the groove element 130.

As shown in FIGS. 6B and 6C, one or more portions of the protrusion 165 may include a ledge member 154. Alternatively, the entirety of the protrusion 165 includes a ledge member 154. The ledge member 154 is designed to enhance the strength of the coupling between the lens 112 and the frame 110. The coupling of the ledge member 154 within the groove element of the frame 110 is described hereinafter in reference to FIG. 7. The ledge member(s) 154 may be positioned on the protrusion 165 in any suitable manner. In preferred embodiments, the protrusion 165 includes a ledge member 154 along certain portions of the protrusion. For example, the ledge member 154 may be positioned only on the protrusion 165 at the top and bottom of the gasket 126, or only on the protrusion 165 at the sides of the gasket 126. As shown in FIG. 6B, ledge members 154 are placed along the top, bottom, and sides of the protrusion 165, but are not placed on the corners of the protrusion 165. FIG. 6C illustrates a cross-section L1 of the gasket 126 at portion of the protrusion 165 that includes a ledge member 154. FIG. 6D illustrates a cross-section L2 of the gasket 126 at a portion of the protrusion 165 that does not include a ledge member 154.

The one or more portions of the protrusion 165 without a ledge member 154 form removal points 152. The removal points 152 are positions where the gasket 126 is not as tightly sealed to the frame 110 (due to absence of the ledge member), and thus require application of less pressure to break the seal between the gasket 126 and the frame 110 (e.g., when one would like to remove the lens 112 from the frame 110). As shown in FIG. 6B, the removal points 152 are placed on the corners of the gasket 126. By placing the removal points 152 on the corners, a user is able to apply outward pressure to the lens at one or more of those corners to break the seal and remove the lens 112. However, the gasket can be designed to form removal points 152 elsewhere.

FIG. 7 highlights the tongue and groove seal of the gasket 126 and the frame 110. As discussed, the tongue and groove seal provides a 360° point of contact seal between the gasket 126 and the frame 110 thereby sealing the lens 112 to the frame 110. FIG. 7 illustrates a cross-section of the goggle system 100 with the lens 112 coupled to the frame 110 via the gasket 126. The cross-section is taken across line y of the goggle system, as shown FIG. 1. The back surface 218 of the gasket 126 mates with the front surface 134 of the frame 110. The front surface 224 of the gasket 126 is coupled to the lens 112 such that a portion of the lens 112 extends beyond the gasket 126, thereby forming an overhang 190. When the lens 112 is coupled to the frame 110, the overhang 190 of the lens 112 covers the front surface 134 of the frame 110, and the outer edge 114 of the lens 112 is substantially flush with the outer edge 115 of the frame 110.

In order to seal the lens 112 to the frame 110 via the gasket 126, the gasket 126 of the lens 112 is pressed into the groove member 130 of the frame 110. Specifically, the protrusion 165 of the gasket 126 enters the channel 164 of the frame's groove member 130. In certain embodiments and as shown, the groove member 130 includes a tab 182 within the channel 164. When the protrusion 165 enters the groove member 130, a ledge member 154 of the protrusion 165 is pushed past the tab 182, and then secured into the channel 164. The tab 182 secures the coupling between the gasket 126 and the frame 110 by holding the ledge member 154 within the channel 164. The tab 182 and/or a wall of the channel 164 are flexible to allow the ledge member 153 to enter the channel 164 and past the tab 182 with use of pressure. The flexibility of the tab and/or a wall of the channel 164 can vary depending on the desired pressure required to remove the lens 112 from the frame 110. When the lens 112 is sealed to the frame 110 via the gasket 126, the opening 150 is closed.

Although not shown in FIG. 7, the goggle system 100 may also include removal points 152 along portions of the seal between the gasket and the frame. The removal points 152 are best shown in FIG. 6B. According to certain embodiments, a cross-section of the goggle system 100 that includes a removal point 152 is substantially the same as the cross-section of FIG. 7 except that the gasket 126 does not include ledge member 154 at the removal point(s) 152. According to other embodiments, a cross-section of the goggle system 100 that includes a removal point 152 is substantially the same as the cross-section of FIG. 7 except that the gasket 126 does not include a ledge member 154 and the groove member 130 does not include tab 182.

The lens 112 can be removed from the frame 110 of the sealed goggle system by applying outward pressure on the lens 112 (e.g., away from the frame). In preferred embodiments, the goggle system includes one or more removal points 152 that require minimal pressure to remove the lens 112 from the frame 110. The removal points 152 are best shown in FIG. 6B. The lens 112 can be removed while the user is wearing the goggle system 100, or with the goggle system 100 removed. The lens 112 can be sealed to the frame by applying forward pressure on the lens 112 against the frame 110 (e.g., towards the frame). The lens 112 can be sealed to the frame 110 of the goggle system 100 while wearing or not wearing the goggle system 100.

A benefit of the current invention is that a user is able to easily remove and replace a lens 112 of the goggle system 100 to suit their particular activity. For example, a user may like to use shaded lens during the day time, but switch to a clear lens at night. In a similar vein, a user may like to change the lens for a different UV protection setting. In another example, a user may want to switch from a prescription lens to a non-prescription lens. In a further example, a user may want to switch from a polarized lens to a non-polarized lens. In addition, the user may simply want to remove a lens for a moment without having to remove the frame from his/her eyes, and then place the same lens back into the frame. Regardless of why or what lens a user would like to use, the goggle system 100 of the invention simply requires a user to remove a lens with application of pressure, and to place a lens on the frame by application of pressure.

The frame 110 of the goggle system 100 can be formed from any suitable material (such as polymeric materials). Exemplary materials include nylon, rubber, urethane, and propionate. In preferred embodiments, the frame 110 of the goggle system is flexible. Flexibility allows the frame to comfortably conform to a user's face to form a seal. In certain embodiments, the frame is a mold injected thermo-plastic urethane frame. As discussed, the frame may be coupled to a foam padding. The foam padding can be of various thickness, and may be formed from polyethylene. In certain embodiments, the frame is molded (e.g., by injection molded).

The goggle system 100 of the invention can be a single lens or a duel lens system. In a single lens system, a single lens is coupled to a front surface of the gasket. In a dual lens system, a first lens is coupled to a front surface of the gasket, and a second lens is coupled to the back surface of the gasket. One or more vents can be placed between the first and the second lens for ventilation.

The lens 112 (whether single or dual) of the goggle system can be formed from any suitable material. In addition, the lens can be a variety of different hues/tints for various visual light transmissions, include UV protection of various spectrums, include scratch resistant technology, and/or include a prescription. In addition, the lenses can be polarized or photochromatic. A lens may also include a reflective coating to reduce glare and/or an anti-fog coating. According to certain embodiments, the lens 112 is more rigid than the frame. Suitable materials for the lens include polymers and nylons. In one embodiment, the lens is a polycarbonate lens. The polymeric material may be of any suitable type, for example, it may include a thermoplastic or thermoset material. A material of the diallyl glycol carbonate type, for example CR-39 (PPG Industries) may be used. The polymeric article may be formed from cross-linkable polymeric casting compositions. The polymeric material may include a dye, preferably a photochromic dye, which may, for example, be added to the monomer formulation used to produce the polymeric material.

The gasket can be formed from any suitable material. In certain embodiments, the gasket is molded (e.g. by injection molding). The lens can be co-molded to the gasket, or the gasket can be formed as part of the lens. Suitable materials for the gasket include polymers, nylons, rubbers, etc. In certain embodiments, the gasket is a polycarbonate material.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A goggle system, comprising
a frame defining an opening and comprising a forward surface that circumscribes the opening, wherein the forward surface comprises a groove element along the entirety of the forward surface; and
a removable lens comprising a gasket configured to mate with the groove element to form a continuous seal between the frame and the removable lens.

2. The goggle system of claim 1, wherein the gasket comprises a tongue element.

3. The goggle system of claim 2, wherein the tongue element comprises a protrusion and a ledge member along a first portion of the protrusion.

4. The goggle system of claim 3, wherein a second portion of the protrusion does not include a ledge member and forms a removal point between the lens and the frame when coupled.

5. The goggle system of claim 4, wherein application of pressure to the removal point releases the seal between the removable lens and the frame.

6. The goggle system of claim 4, wherein the protrusion comprises two or more removal points.

7. A goggle system, comprising
a frame defining an opening and comprising a forward surface, the forward surface faces away from a user when the user wears the goggle system, circumscribes the opening of the frame, and comprises an outer perimeter, and
a lens configured to removably couple to the frame such that an outer perimeter of the lens is substantially flush with the outer perimeter of the frame's front surface, thereby closing the opening.

8. The goggle system of claim 7, wherein the forward surface of the frame further comprises a groove element.

9. The goggle system of claim 8, wherein the lens comprises a tongue element configured to mate with the groove element of the frame.

10. The goggle system of claim 9, wherein the tongue element is a part of a gasket.

11. The goggle system of claim 8, wherein the groove element forms along the entirety of the forward surface.

12. The goggle system of claim 9, wherein the tongue element, when mated with the groove element, forms a continuous seal between the frame and the removable lens.

13. The goggle system of claim 12, wherein the tongue element comprises a protrusion and a ledge member along a first portion of the protrusion.

14. The goggle system of claim 13, wherein a second portion of the protrusion does not include a ledge member and forms a removal point between the lens and the frame when coupled.

15. The goggle system of claim 14, wherein application of pressure to the removal point releases the seal between the removable lens and the frame.

16. The goggle system of claim 14, wherein the protrusion comprises two or more removal points.

17. A goggle system, comprising
a frame defining an opening and comprising a forward surface that circumscribes the opening, wherein the forward surface comprising a groove element along the entirety of the forward surface;
a gasket comprising a tongue element configured to removably mate with the groove element to form a continuous seal between the gasket and the frame; and
a lens coupled to gasket such that, when the gasket mates with the frame, the lens closes the opening.

18. The goggle system of claim 17, wherein the tongue element comprises a protrusion and a ledge member along a first portion of the protrusion.

19. The goggle system of claim 18, wherein a second portion of the protrusion does not include a ledge member and forms a removal point between the lens and the frame when coupled.

20. The goggle system of claim 19, wherein application of pressure to the removal point releases the seal between the removable lens and the frame.

21. The goggle system of claim 19, wherein the protrusion comprises two or more removal points.

22. A goggle system, comprising
a frame defining an opening and comprising a forward surface that circumscribes the opening, wherein the forward surface comprises a channel around the entirety of the forward surface; and
a removable lens comprising a protrusion that includes a ledge member along a first portion of the protrusion, wherein the protrusion configured to couple with the channel to form a continuous seal between the frame and the removable lens.

23. The goggle system of claim 22, wherein a second portion of the protrusion does not include a ledge member and forms a removal point between the removable lens and the frame when sealed.

24. The goggle system of claim 23, wherein application of pressure to the removal point release the seal between the removable lens and the frame.

25. The goggle system of claim 24, wherein the protrusion comprises two or more removal points.
